(19) United States
(12) Patent Application Publication
(10) Pub. No.: US 2026/0242534 A1
(43) Pub. Date: Aug. 20, 2026

(54) PRODUCING SPECIAL ISOCYANATES VIA CO-PHOSGENATION

(71) Applicant: Covestro Deutschland AG, Leverkusen (DE)

(72) Inventors: Frank Richter, Leverkusen (DE); Ruediger Borrmann, Köln (DE); Tim Loddenkemper, Dormagen (DE); Dieter Schade, Bergisch Gladbach (DE)

(21) Appl. No.: 18/876,695

(22) PCT Filed: Jun. 20, 2023
(86) PCT No.: PCT/EP2023/066617

§ 371 Date: Dec. 19, 2024

(30) Foreign Application Priority Data
Jun. 22, 2022 (EP) 22180309.1

(51) Int. Cl. C08G 18/77; B01J 19/00; B01J 19/18; C07C 263/10; C07D 307/22; C07D 493/04; C09D 175/04; C09J 175/04

(52) U.S. Cl. CPC C08G 18/771; B01J 19/0006; B01J 19/18; C07C 263/10; C07D 307/22; C07D 493/04; C09D 175/04; C09J 175/04; B01J 2219/00186

(57) ABSTRACT

The invention relates to a method for producing isocyanates via phosgenation, in particular gas-phase phosgenation (GPP) or liquid-phase phosgenation (FPP), of the corresponding amines, wherein an amine mixture formed of at least one first amine and at least one second amine, that is different from the first amine, is reacted with phosgene to form a reaction mixture, in which, in particular, a higher yield and/or good purity (in the form of a lowering of the content of acid chlorine compounds (AC value) and/or of hydrolysable chlorine (HC value)) is achieved for the corresponding isocyanates obtained in the production process. The invention also relates to a product that is/can be obtained using the method according to the invention, preferably a product that is/can be obtained directly using the method according to the invention, as well as the use of said product and/or the isocyanate or isocyanate mixture which is/can be obtained using the method according to the invention. The invention further relates to a phosgenation system for carrying out the method according to the invention.

The invention relates to a process for producing isocyanates by phosgenation, in particular gas-phase phosgenation (GPP) or liquid-phase phosgenation (LPP), of the corresponding amines, wherein an amine mixture formed of at least one first amine and at least one second amine different from the first amine is reacted with phosgene to afford a reaction mixture in which, in particular, a higher yield and/or good purity (in the form of a reduction in the content of acidic chlorine compounds (AC value) and/or of hydrolyzable chlorine (HC value)) is achieved for the corresponding isocyanates obtained in the production process. The invention also relates to a product obtainable or obtained by the process of the invention, preferably a product directly obtainable or obtained by the process of the invention, and to the use of said product and/or isocyanate or isocyanate mixture obtained or obtainable by the process of the invention. The invention further relates to a phosgenation plant for performance of the process of the invention.

Isocyanates, in particular diisocyanates, in some cases also low-molecular-weight triisocyanates, as well as the higher-molecular-weight polyaddition products with terminal isocyanate groups obtainable from said compounds, are valuable raw materials for the production of polyurethanes. It is industrially advantageous to produce isocyanates by phosgenation of the parent amines either in the condensed phase (liquid-phase phosgenation, hereinafter LPP) or in the gas phase (gas-phase phosgenation, hereinafter GPP). The latter is practicable only in the case of amines that can be vaporized at the chosen pressure without decomposition. In general, it has been found that many isocyanates that are not producible by LPP, or producible only in poor yields or with unsatisfactory purity, can be better produced by GPP. This is true in particular of diisocyanates bearing sensitive functional groups in their molecular structure, for example ether bridges (cf. EP-A 764633 and sources cited therein).

Although some isocyanates containing ether groups can be produced by GPP in good yields and high purity, this is by no means true of all industrially relevant species in this substance class. For instance, the acidity or content of acidic chlorine compounds (hereinafter also: AC, AC value, AC content) and the hydrolyzable chlorine content (hereinafter also: HC, HC value, HC content) of, for example, 1,5-diisocyanato-3-oxapentane (hereinafter OBDI) is not sufficiently low to permit problem-free further processing of this diisocyanate in typical polyurethane applications (see comparative examples). Other examples are (poly)cyclic-ether-group-containing amines with cyclic ether building blocks, for example sugar derivatives of the furan, dihydrofuran, tetrahydrofuran, sorbitol, mannitol and iditol series (isohexides), it being immaterial whether the amino groups are attached to the (poly)cycle directly or via further bridging atoms. Distillative removal of HC-bearing impurities is for many of these compounds difficult, if not impossible, particularly for OBDI.

The object of the present invention was therefore to provide a process for producing isocyanates by phosgenation (gas-phase and/or liquid-phase phosgenation) of the corresponding amines and also a phosgenation plant for performance of this process. In particular, it was a further object of the present invention to convert amines that are “problematic” with regard to phosgenation (hereinafter also: “amine(s) 1” or “first amine”) into the corresponding isocyanates (hereinafter also: “isocyanate(s) 1”) in an industrially efficient manner. In particular, a high yield and/or good purity should be achieved in the form of a reduction in the content of acidic chlorine compounds (AC value) and/or of hydrolyzable chlorine (HC value) of the corresponding isocyanates obtained in the production process.

This object was achieved by a process for producing isocyanates by phosgenation of the corresponding amines, wherein an amine mixture formed of at least one first amine and at least one second amine different from the first amine is reacted with phosgene to afford a reaction mixture, characterized in that either
the first amine contains at least one ether group; and
the second amine is selected from the group comprising or consisting of aminoalkanes that have 5 to 15 hydrocarbon atoms and do not contain ether groups;
or
the first amine contains at least one cyclic ether group; and
the second amine contains at least one open-chain ether group and no cyclic ether groups.

The invention also relates to the use of an amine mixture formed of at least one first amine and at least one second amine different from the first amine in a process for producing the corresponding isocyanates by phosgenation according to the process of the invention for reducing the content of acidic chlorine compounds and/or the content of hydrolyzable chlorine of the corresponding isocyanates obtained in the production process.

The invention further relates to a product obtainable or obtained by the process of the invention, preferably directly obtainable or obtained by the process of the invention.

In addition, the invention relates to the use of the product of the invention and/or of the isocyanate or isocyanate mixture obtained or obtainable by the process of the invention as a component for the production of polyurethanes, in particular polyurethane foams, polyurethane coatings, and polyurethane adhesives, of pharmaceutical products, in particular active substances, and also of auxiliaries, in particular auxiliaries for wet-strength finishing of paper and other cellulose products, emulsifiers, and thickeners.

The invention further relates to a phosgenation plant for performance of the process of the invention, comprising or consisting of:
(a) at least one phosgene apparatus for the continuous supply of phosgene, optionally in combination with an inert substance,
(b) optionally an inert substance apparatus for the supply of an inert substance,
(c) at least one amine apparatus for the continuous supply of an amine mixture, optionally in combination with an inert substance, and
(d) a phosgenation reactor for mixing the phosgene and the amine mixture and optionally the inert substance and reacting the amine mixture with the phosgene, characterized in that the amine apparatus comprises a first reservoir vessel for a first amine and a second reservoir vessel for a second amine, and also a metering device for independently variable adjustment of the mass fractions of the first amine and the second amine, and optionally a mixing device for mixing the first amine and the second amine to form the amine mixture.

Surprisingly, it was found that mixtures of amines 1 (“first amine”) with amines that are unproblematic with regard to phosgenation (hereinafter also: “amines 2” or “second amine”) can be converted into the corresponding mixtures of isocyanate(s) 1 and isocyanate(s) 2 much more smoothly than is the case with phosgenation of the amines 1 on their own. This process of the invention is also referred to hereinafter as co-phosgenation, or CoPg for short. The resulting isocyanates 2 are at least equivalent in yield and quality (particularly in the content of acidic chlorine compounds (AC value) and/or of hydrolyzable chlorine compounds (HC value)) to those resulting from phosgenation of the amines 2 on their own. However, a particular surprise is that the diisocyanates 2 obtainable from the amines 2 by the process of the invention are also sometimes produced in significantly higher purity than is the case with phosgenation on their own under otherwise comparable conditions. Further details thereon are given in the exemplary embodiments. In addition, it was found that amines having open-chain ether groups are relatively unproblematic with regard to phosgenation by comparison with amines having cyclic ether groups. Here too, it was found in the process of the invention that an amine mixture formed of an amine having at least one cyclic ether group and an amine having exclusively open-chain ether groups show the same improvements in yield and quality (particularly in the content of acidic chlorine compounds (AC value) and/or of hydrolyzable chlorine compounds (HC value)). In the context of the invention, the content of acidic chlorine compounds and the content of hydrolyzable chlorine was determined in accordance with the ISO 15028:2014 standard.

Although the phosgenation of isomer mixtures, for example of 2,4- and 2,6-diaminotoluene to the industrially established TDI and also the phosgenation of isomer and homolog mixtures of the amines resulting from the aniline-formaldehyde condensation (MDA-->MDI) are well-known examples of the phosgenation of mixtures of different individual chemicals, the rationale for this approach is purely that the production of the parent amines gives rise to isomer and/or homolog mixtures for which separation into their individual constituents appears uneconomic or technically impractical. The option employed here is instead for the substances, which are more readily obtainable in pure form by crystallization (2,4-TDI, so-called “T 100”) or distillation (monomeric MDI, e.g. 4,4′-bis(isocyanatophenyl)methane, 2,4′-bis(isocyanatophenyl)methane, and 2,2′-bis(isocyanatophenyl)methane and any mixtures of the above three individual compounds, hereinafter: 2-ring MDI)) to be isolated from the isocyanate mixtures rather than carrying out a corresponding separation of the amine mixtures beforehand. In the aliphatic series, IPDA and ring-hydrogenated 2-ring MDA (established acronyms: H12MDA, PACM) are examples of the mixtures of diamine regio- and/or stereoisomers that inevitably occur, which after phosgenation are converted into the corresponding isomer mixtures of the diisocyanates IPDI and/or H₁₂-MDI respectively. In the latter case, separation of the pure trans-trans-4,4′ isomer is also possible by crystallization.

A deliberately provided mixture of two (or more) chemically completely different amines from completely different production processes for the purpose of phosgenation has up to now been described only in DE 2249459. In this case it is essential to the invention to use an aromatic amine 2 (in the sense of the present application) as the “base amine” for the production of aliphatic isocyanates from the corresponding amines, in order to avoid having to build a costly separate plant for the aliphatic isocyanates, which are usually technically required in significantly smaller volumes. The solution to the abovementioned technical problems was neither provided by DE 2249459 nor is it solvable according to the teaching therein, as can be seen from the HC contents of the isocyanates described in the examples of DE 2249459 (0.1% and above). As example 1 of the present document shows, the process of DE 2249459 is also not capable of overcoming the problems in the production of specialty isocyanates in the context of the present application.

Also surprising, particularly in view of the average free path length between the different molecules in the gas phase on the one hand and in view of the often-high dilution in the liquid phase on the other hand, is that CoPg of the first and second amine (amines 1 and 2) has some kind of beneficial influence on the overall operation aside from the obvious economic advantages discussed in DE 2249459, since a second plant is not needed for the production of specialty isocyanates with lower tonnage than is customary for MDI production, for example.

The invention relates to a process for producing isocyanates by phosgenation of the corresponding amines, wherein an amine mixture formed of at least one first amine and at least one second amine different from the first amine is reacted with phosgene to afford a reaction mixture, characterized in that either
the first amine contains at least one ether group; and
the second amine is selected from the group comprising or consisting of aminoalkanes that have 5 to 15 hydrocarbon atoms and do not contain ether groups;
or
the first amine contains at least one cyclic ether group; and
the second amine contains at least one open-chain ether group and no cyclic ether groups.

The phosgenation is preferably a gas-phase phosgenation (GPP) or liquid-phase phosgenation (LPP). The mixing ratio of the first and second amines (amines 1 and 2) may vary within wide limits in the process of the invention (20:1 to 1:20). Preferably, the mixing ratio of the first amine to the second amine is 1:9 to 9:1, more preferably 2:8 to 8:2. Although high proportions of amine 1 in the amine mixture obviously lead to a higher yield of the isocyanate 1 resulting therefrom, for production of particularly pure isocyanates 1 it can also be preferable for the proportion of amine 1 undergoing CoPg as a mixture with amine 2 not to be too high. In gas-phase phosgenation it is also possible to alter the ratio of amine 1 to amine 2 (first amine to second amine) in the course of the reaction, as far as metering in exclusively amine 2 in the start-up phase and ultimately then metering in exclusively amine 1 once a stable operating state (steady state) has been attained. In this specific embodiment of the process, use is made of the fact that, particularly during start-up of the gas-phase phosgenation, critical phases are often passed through that are better managed with the amine 2 than with amine 1 and in which metering in the latter on its own in the start-up phase would lead to abandonment of the experiment.

In the selection of amine 1 and 2 care can be taken to ensure that the resulting isocyanates 1 and 2 are easily separable from one another, for example by distillation, extraction or crystallization. However, it is also possible to carry on using the mixtures of isocyanates 1 and 2—usually after distillative removal of coloring and high-boiling impurities—in the form of the mixture in which they occur in the process of the invention.

This approach is expressly excluded in DE 2249459, since it is stressed therein that it is essential to the invention for the (two) isocyanates to be separable at the end of the process (cf. DE 2249459, p. 4, final paragraph and claims 1-3).

In particular, the first amine containing at least one ether group, the first amine containing at least one cyclic ether group, the second amine comprising or consisting of aminoalkanes having 5 to 15 hydrocarbon atoms and/or the second amine containing at least one open-chain ether group and no cyclic ether groups is a diamine.

The first amine containing at least one ether group preferably includes a structure of the general formula X—(—R¹—NHₘ)ₙ, where
X is H, NHₘ or C(R²)ₚ;
R¹ is optionally substituted and/or heteroatom-containing aliphatic, (cyclo)aliphatic or aromatic radicals having preferably up to 10 carbon atoms, where R¹ contains at least one ether group;
R² is H or optionally substituted and/or heteroatom-containing aliphatic, (cyclo)aliphatic or aromatic radicals having preferably up to 10 carbon atoms;
m is 1 or 2;
n is 1, 2, or 3; and
p is 1, 2 or 3.

In a preferred embodiment, the first amine containing at least one ether group is selected from the group comprising or consisting of (tetrahydrofuran-2,5-diyl)dimethanamine (TEFUDA), 3,6-diaminohexahydrofuro[3,2-b]furan (e.g. IDDA and/or ISODA), 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine; di(aminoethyl) ether, especially 2,2′-oxybis(ethan-1-amine), 1,1′-oxybis(propan-2-amine), 2-(2-aminoethoxy)propan-1-amine, 2,2′-oxybis(propan-1-amine), and 2-(2-aminopropoxy)propan-1-amine; di(aminopropyl) ether or mixtures thereof. The first amine containing at least one cyclic ether group is preferably selected from the group comprising or consisting of (tetrahydrofuran-2,5-diyl)dimethanamine (TEFUDA), 3,6-diaminohexahydrofuro[3,2-b]furan (e.g. IDDA and/or ISODA), 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine or mixtures thereof. It is further preferable when the second amine comprising or consisting of aminoalkane having 5 to 15 hydrocarbons is selected from the group comprising or consisting of 1,5-diaminopentane; 1,6-diaminohexane; 1-amino-3,5,5-trimethyl-5-aminomethylcyclohexane; amino-[(aminocyclohexyl)methyl]cyclohexane, especially 4,4′-methylenebis-(cyclohexan-1-amine), 2-((4-aminocyclohexyl)methyl)cyclohexan-1-amine, and 2,2′-methylenebis(cyclohexan-1-amine); 1,3-bis(aminomethyl)cyclohexane; 1,4-bis(aminomethyl)cyclohexane; diaminocyclohexane, especially cyclohexane-1,2-diamine, cyclohexane-1,3-diamine, and cyclohexane-1,4-diamine; methyldiaminocyclohexane, especially 4-methylcyclohexane-1,3-diamine and 2-methylcyclohexane-1,3-diamine; 4,4′-methylenebis(2-methylcyclohexan-1-amine); or mixtures thereof. However, a number of other amines may also be suitable as “amines 2”, which can be easily determined through simple preliminary tests.

The second amine containing at least one open-chain ether group and no cyclic ether groups is preferably selected from the group comprising or consisting of di(aminoethyl) ether, di(aminopropyl) ether, 1,8-diamino-1,5,8-trimethyl-3,6-dioxaoctane, 1,11-diamino-1,5,8,11-tetramethylundecane, 1,8-diamino-3,6-dioxaoctane, 1,10-diamino-4,7-dioxadecane, 1,12-diamino-4,9-dioxadodecane, 1,14-diamino-3,10-dioxatetradecane, 1,13-diamino-4,7,10-trioxatridecanane, 1,7-diamino-2,6-dioxa-4-aminomethoxy-heptane, 1-amino-2-oxa-3,3-bis(aminomethoxy)hexane, 1,9-diamino-3,7-dioxa-5-(1-amino-2-ethoxy)-nonane, 1-amino-3-oxa-4,4-bis(1-amino-2-ethoxy)heptane, 1,11-diamino-4,8-dioxa-6-(1-amino-5-oxabutyl)undecane, 1-amino-4-oxa-5,5-bis(1-amino-5-oxabutyl)octane or mixtures thereof. The limiting factor with regard to the usability of mono- and polyamines containing ether groups in the CoPg of the invention in the specific embodiment of gas-phase phosgenation is to be seen neither in the number of (primary) amino groups nor in the number of ether bonds, but is determined solely by the vaporability under the applied pressure. In the case of particularly high-boiling compounds, it may therefore be advantageous to introduce them into the gas-phase phosgenation in the form of azeotropes with other substances or to use a carrier gas for feeding the amine mixture into the reaction space. In this case, amine 2 can have a promoting effect.

In addition, it is preferable that the mass fraction
of the first amine is 5.0% to 95.0% by weight, preferably 10.0% to 80.0% by weight and
of the second amine is 95.0% to 5.0% by weight, preferably 90.0% to 20.0% by weight, more preferably 80.0% to 20.0%,
   based on the total weight of the amine mixture.

The molar ratio of phosgene to the amino groups of the amines in the amine mixture is preferably ≥1:1 to ≤5:1, preferably >1:1 to ≤3:1, more preferably >1:1 to ≤2:1.

It is preferable when an inert substance is additionally employed in the reaction, the inert substance being selected from the group comprising or consisting of:
inert gases, in particular nitrogen, argon or a mixture thereof;
inert solvents, in particular aromatic hydrocarbons, preferably chlorobenzene, o-dichlorobenzene, toluene, xylene or mixtures thereof;
or mixtures of the aforementioned inert gases and inert solvents.

Preferably, the temperature
in the condensed-phase reaction is firstly between −40° C. and the boiling point of the solvent used or of the lowest-boiling input material at the pressure established in the reaction and
in the gas-phase phosgenation is between the boiling point of the reactant having the highest boiling point T1 at system pressure and 600° C., preferably between T1 and 550° C., more preferably between T1 and 500° C.

It is also preferable that the isocyanate corresponding to the first amine and/or the isocyanate corresponding to the second amine is separated from the reaction mixture, separation preferably being effected by distillation, in particular thin-film distillation, extraction, crystallization, recrystallization or a combination thereof, and with the respective isocyanates obtained separately or as an isocyanate mixture. It is further preferable that the isocyanate corresponding to the first amine and the isocyanate corresponding to the second amine is separated from the reaction mixture, separation preferably being effected by distillation, in particular thin-film distillation, extraction, crystallization, recrystallization or a combination thereof, and with the respective isocyanates obtained separately or as an isocyanate mixture. The content of acidic chlorine compounds (also referred to as AC value, AC content or acidity) in the separated isocyanate(s) or in the isocyanate mixture is from 1 to 100 ppm, preferably from 2 to 80 ppm, more preferably from 5 to 50 ppm, determined according to the ISO 15028:2014 standard. The content of hydrolyzable chlorine (also referred to as HC value or HC content) in the separated isocyanate(s) or in the isocyanate mixture is preferably from 1 to 500 ppm, preferably from 5 to 100 ppm, determined according to the ISO 15028:2014 standard.

In a preferred embodiment, the invention relates to a process for producing isocyanates by phosgenation of the corresponding amines, wherein an amine mixture formed of at least one first amine and at least one second amine different from the first amine is reacted with phosgene to afford a reaction mixture, characterized in that either
the first amine contains at least one ether group; and
the second amine is selected from the group comprising or consisting of aminoalkanes that have 5 to 15 hydrocarbon atoms and do not contain ether groups;
or
the first amine contains at least one cyclic ether group; and
the second amine contains at least one open-chain ether group and no cyclic ether groups,
   wherein the first amine containing at least one ether group is selected from the group comprising or consisting of (tetrahydrofuran-2,5-diyl)dimethanamine, 3,6-diaminohexahydrofuro[3,2-b]furan, 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine; di(aminoethyl) ether, especially 2,2′-oxybis(ethan-1-amine), 1,1′-oxybis(propan-2-amine), 2-(2-aminoethoxy)propan-1-amine, 2,2′-oxybis(propan-1-amine) and 2-(2-aminopropoxy)propan-1-amine; di(aminopropyl) ether or mixtures thereof and/or the first amine containing at least one cyclic ether group is selected from the group comprising or consisting of (tetrahydrofuran-2,5-diyl)dimethanamine, 3,6-diaminohexahydrofuro[3,2-b]furan, 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine or mixtures thereof, and the second amine comprising or consisting of aminoalkane having 5 to 15 hydrocarbon atoms is selected from the group comprising or consisting of 1,5-diaminopentane; 1,6-diaminohexane; 1-amino-3,5,5-trimethyl-5-aminomethylcyclohexane; amino-[(aminocyclohexyl)methyl]cyclohexane, especially 4,4′-methylenebis(cyclohexan-1-amine), 2-((4-aminocyclohexyl)methyl)cyclohexan-1-amine, and 2,2′-methylenebis(cyclohexan-1-amine); 1,3-bis(aminomethyl)cyclohexane, 1,4-bis(aminomethyl)cyclohexane; diaminocyclohexane, especially cyclohexane-1,2-diamine, cyclohexane-1,3-diamine, and cyclohexane-1,4-diamine; methyldiaminocyclohexane, especially 4-methylcyclohexane-1,3-diamine and 2-methylcyclohexane-1,3-diamine, 4,4′-methylenebis(2-methylcyclohexan-1-amine); or mixtures thereof, and the second amine containing at least one open-chain ether group and no cyclic ether groups is selected from the group comprising or consisting of di(aminoethyl) ether, di(aminopropyl) ether, 1,8-diamino-1,5,8-trimethyl-3,6-dioxaoctane, 1,11-diamino-1,5,8,11-tetramethylundecane, 1,8-diamino-3,6-dioxaoctane, 1,10-diamino-4,7-dioxadecane, 1,12-diamino-4,9-dioxadodecane, 1,14-diamino-3,10-dioxatetradecane, 1,13-diamino-4,7,10-trioxatridecanane, 1,7-diamino-2,6-dioxa-4-aminomethoxy-heptane, 1-amino-2-oxa-3,3-bis(aminomethoxy)hexane, 1,9-diamino-3,7-dioxa-5-(1-amino-2-ethoxy)-nonane, 1-amino-3-oxa-4,4-bis(1-amino-2-ethoxy)heptane, 1,11-diamino-4,8-dioxa-6-(1-amino-5-oxabutyl)undecane, 1-amino-4-oxa-5,5-bis(1-amino-5-oxabutyl)octane or mixtures thereof. This embodiment may be freely combined with the other embodiments provided the contrary is not apparent from the context.

The invention further relates to the use of an amine mixture formed of at least one first amine and at least one second amine different from the first amine in a process for producing the corresponding isocyanates by phosgenation according to a process as claimed in any of claims 1 to 13 for reducing the content of acidic chlorine compounds and/or the content of hydrolyzable chlorine of the corresponding isocyanates obtained in the production process.

The present invention further provides a product obtainable or obtained by the process of the invention, preferably directly obtainable or obtained by the process of the invention.

The invention also provides for the use of the product of the invention and/or of the isocyanate or isocyanate mixture obtained or obtainable by the process of the invention as a component for the production of polyurethanes, in particular polyurethane foams, polyurethane coatings, and polyurethane adhesives, of pharmaceutical products, in particular active substances, and also of auxiliaries, in particular auxiliaries for wet-strength finishing of paper and other cellulose products, emulsifiers, and thickeners.

The GPP process of the invention is in its main features executed by means of a prior art method known per se, for example according to the teaching of EP 0 570 799 or EP 0676392 and process variants cited therein. For this purpose, the coreactants are introduced into suitable reactors close to or above the boiling temperature of the starting amine (mixture), mixed and reacted with each other. Depending on the selected pressure, the temperatures for this are between 10° and 600° C., preferably between 15° and 500° C. The process is carried out in a pressure range of between 10 mbar and 5 bar, preferably 200 mbar and 3 bar. The reaction components in the gas-phase phosgenation can be supplied with or without the use of inert additives such as carrier gases for the phosgenation reaction. Suitable carrier gases include nitrogen, argon or other inert gases, as well as vapors of technically available solvents, such as chlorobenzene, dichlorobenzene, xylenes, chloronaphthalenes, decahydronaphthalene, etc.

The isocyanate mixtures of the invention are then obtained by cooling the gas stream down to a temperature above the decomposition temperature of the corresponding carbamoyl chloride intermediate. In a preferred embodiment, the isocyanate corresponding to the first amine and/or the isocyanate corresponding to the second amine is then separated from the reaction mixture, separation preferably being effected by distillation, in particular thin-film distillation, extraction, crystallization, recrystallization or a combination thereof, and with the respective isocyanates obtained separately or as an isocyanate mixture. Particularly preferably, the isocyanate corresponding to the first amine and the isocyanate corresponding to the second amine are separated from the reaction mixture. It is also preferable that separation by distillation is optionally followed by recrystallization.

The process products of the invention are valuable raw materials for the production of polyurethanes, adhesives, coating agents, oligomeric isocyanate modification products such as uretdione-, isocyanurate-, carbodiimide-, biuret-, urethane- and allophanate-containing polyisocyanates, auxiliaries such as those used for the wet-strength finishing of paper and other cellulose products, as emulsifiers, thickeners, etc., as a raw material for the production and/or formulation of active substances, pharmaceuticals, etc.

Finally, the invention further relates to a phosgenation plant for performance of the process of the invention, comprising or consisting of:
(a) at least one phosgene apparatus for the continuous supply of phosgene, optionally in combination with an inert substance,
(b) optionally an inert substance apparatus for the supply of an inert substance,
(c) at least one amine apparatus for the continuous supply of an amine mixture, optionally in combination with an inert substance, and
(d) a phosgenation reactor for mixing the phosgene and the amine mixture and optionally the inert substance and reacting the amine mixture with the phosgene,
characterized in that the amine apparatus comprises a first reservoir vessel for a first amine and a second reservoir vessel for a second amine, and also a metering device for independently variable adjustment of the mass fractions of the first amine and the second amine, and optionally a mixing device for mixing the first amine and the second amine to form the amine mixture.

The phosgene stream apparatus, the optional inert substance stream apparatus, the amine stream apparatus and the phosgenation reactor may mutually independently comprise further apparatuses useful for the operation of the phosgenation plant, for example reservoir vessels for starting materials, and heat exchangers for controlling the temperature of or optionally evaporating starting materials, and devices for processing the reaction mixture on completion of the reaction of amine with phosgene.

The metering device may be for example a metering pump or a control valve. The optional mixing device may, for example, be a T-piece in the pipeline, a mixing chamber, a static mixer or a pump, especially mixing pump, or stirred vessel. However, the amine streams from the metering devices can also be mixed in the evaporator. An evaporator is used in the case of liquid amines in particular.

The amine stream apparatus preferably comprises, in at least one of the amine-conducting pipelines, at least one flange connection prepared for accommodation of a blank. This enables reliable prevention of amine flow through the corresponding conduit by means of the blank or reliable severing of the corresponding amine conduit.

The phosgene stream apparatus, the optional inert substance stream apparatus and the amine stream apparatus are each connected to the phosgenation reactor via at least one inlet. The connection of the inlets to the phosgenation reactor may, for example, be via simple pipelines, flange connections, smooth jet nozzles, annular gap nozzles or a mixing element. The annular gap nozzle may be equipped here with two or more channels, preferably two in a coaxial arrangement, for the amine stream and the phosgene stream or an inert gas stream, where the amine stream and the phosgene stream do not meet until within the phosgenation reactor. Alternatively, especially in industrial scale application, the amine is added to the phosgenation reactor via an inlet tube, at the end of which there is a central nozzle and which is kept in position by a cover and optional holder/flow straightener, such that an annular space is formed around the inlet tube in the reactor, while the phosgene enters the ring space via a separate inlet and thence reaches the phosgenation reactor via the flow straightener (see also EP1362847 A2). The phosgenation reactor has a mixing zone where the streams meet for the first time, and a reaction zone where the streams are reacted further. The mixing zone and reaction zone are preferably in one apparatus. In addition, it is preferable that the phosgenation reactor is essentially upright, and this is preferably connected at the upper end to the phosgene stream apparatus and the amine stream apparatus.

The phosgenation plant also preferably comprises at least one quench for cooling and at least partial condensation of the reaction mixture, which is connected to the phosgenation reactor, preferably at the lower end, where the quench is designed in particular for introduction of chlorobenzene, o-dichlorobenzene and/or at least one of the isocyanates produced in the phosgenation plant. In particular, the quench has a dedicated collecting vessel, where the collecting vessel is preferably incorporated in or separately connected to the quench.

Preferably, the amine apparatus, the optional separate inert substance apparatus and/or the phosgene apparatus include a heat exchanger for heating the amine mixture, the phosgene and/or optional inert substance.

### EMBODIMENTS

The present invention relates in particular to the following embodiments:

In a first embodiment, the invention relates to a process for producing isocyanates by phosgenation of the corresponding amines, wherein an amine mixture formed of at least one first amine and at least one second amine different from the first amine is reacted with phosgene to afford a reaction mixture, characterized in that either
the first amine contains at least one ether group; and
the second amine is selected from the group comprising or consisting of aminoalkanes that have 5 to 15 hydrocarbon atoms and do not contain ether groups;
or
the first amine contains at least one cyclic ether group; and
the second amine contains at least one open-chain ether group and no cyclic ether groups.

In a second embodiment, the invention relates to a process according to embodiment 1, characterized in that the first amine containing at least one ether group includes a structure of the general formula X—(—R¹—NHₘ)ₙ, where
X is H, NHₘ or C(R²)p
R¹ is optionally substituted and/or heteroatom-containing aliphatic, (cyclo)aliphatic or aromatic radicals having preferably up to 10 carbon atoms, where R¹ contains at least one ether group;
R² is H or optionally substituted and/or heteroatom-containing aliphatic, (cyclo)aliphatic or aromatic radicals having preferably up to 10 carbon atoms;
m is 1 or 2;
n is 1, 2, or 3; and
p is 1, 2 or 3.

In a third embodiment, the invention relates to a process according to embodiment 1 or 2, characterized in that the first amine containing at least one ether group, the first amine containing at least one cyclic ether group, the second amine comprising or consisting of aminoalkanes having 5 to 15 hydrocarbon atoms and/or the second amine containing at least one open-chain ether group and no cyclic ether groups is a diamine.

In a fourth embodiment, the invention relates to a process according to any of the preceding embodiments, characterized in that the first amine containing at least one ether group is selected from the group comprising or consisting of (tetrahydrofuran-2,5-diyl)dimethanamine (TEFUDA), 3,6-diaminohexahydrofuro[3,2-b]furan (e.g. IDDA and/or ISODA), 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine; di(aminoethyl) ether, especially 2,2′-oxybis(ethan-1-amine), 1,1′-oxybis(propan-2-amine), 2-(2-aminoethoxy)propan-1-amine, 2,2′-oxybis(propan-1-amine) and 2-(2-aminopropoxy)propan-1-amine; di(aminopropyl) ether or mixtures thereof and/or the first amine containing at least one cyclic ether group is preferably selected from the group comprising or consisting of (tetrahydrofuran-2,5-diyl)dimethanamine, 3,6-diaminohexahydrofuro[3,2-b]furan (e.g. IDDA and/or ISODA), 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine or mixtures thereof.

In a fifth embodiment, the invention relates to a process according to any of the preceding embodiments, characterized in that the second amine comprising or consisting of aminoalkane having 5 to 15 hydrocarbon atoms is selected from the group comprising or consisting of 1,5-diaminopentane; 1,6-diaminohexane; 1-amino-3,5,5-trimethyl-5-aminomethylcyclohexane; amino-[(aminocyclohexyl)methyl]cyclohexane, especially 4,4′-methylenebis(cyclohexan-1-amine), 2-((4-aminocyclohexyl)methyl)cyclohexan-1-amine, and 2,2′-methylenebis-(cyclohexan-1-amine); 1,3-bis(aminomethyl)cyclohexane; 1,4-bis(aminomethyl)-cyclohexane; diaminocyclohexane, especially cyclohexane-1,2-diamine, cyclohexane-1,3-diamine, and cyclohexane-1,4-diamine; methyldiaminocyclohexane, especially 4-methylcyclohexane-1,3-diamine and 2-methylcyclohexane-1,3-diamine; 4,4′-methylenebis(2-methylcyclohexan-1-amine); or mixtures thereof.

In a sixth embodiment, the invention relates to a process according to any of the preceding embodiments, characterized in that the second amine containing at least one open-chain ether group and no cyclic ether groups is selected from the group comprising or consisting of di(aminoethyl) ether, di(aminopropyl) ether, 1,8-diamino-1,5,8-trimethyl-3,6-dioxaoctane, 1,11-diamino-1,5,8,11-tetramethylundecane, 1,8-diamino-3,6-dioxaoctane, 1,10-diamino-4,7-dioxadecane, 1,12-diamino-4,9-dioxadodecane, 1,14-diamino-3,10-dioxatetradecane, 1,13-diamino-4,7,10-trioxatridecanane, 1,7-diamino-2,6-dioxa-4-aminomethoxy-heptane, 1-amino-2-oxa-3,3-bis(aminomethoxy)hexane, 1,9-diamino-3,7-dioxa-5-(1-amino-2-ethoxy)-nonane, 1-amino-3-oxa-4,4-bis(1-amino-2-ethoxy)heptane, 1,11-diamino-4,8-dioxa-6-(1-amino-5-oxabutyl)undecane, 1-amino-4-oxa-5,5-bis(1-amino-5-oxabutyl)octane or mixtures thereof.

In a seventh embodiment, the invention relates to a process according to any of the preceding embodiments, characterized in that the mass fraction
of the first amine is 5.0% to 95.0% by weight, preferably 10.0% to 80.0% by weight and
of the second amine is 95.0% to 5.0% by weight, preferably 90.0% to 20.0% by weight, more preferably 80.0% to 20.0%,
   based on the total weight of the amine mixture.

In an eighth embodiment, the invention relates to a process according to any of the preceding embodiments, characterized in that the molar ratio of phosgene to the amino groups of the amines in the amine mixture is ≥1:1 to ≤5:1, preferably >1:1 to ≤3:1.

In a ninth embodiment, the invention relates to a process according to any of the preceding embodiments, characterized in that an inert substance is employed in the reaction, the inert substance being selected from the group comprising or consisting of:
inert gases, in particular nitrogen, argon or a mixture thereof;
inert solvents, in particular aromatic hydrocarbons, preferably chlorobenzene, o-dichlorobenzene, toluene, xylene or mixtures thereof;
or mixtures of the aforementioned inert gases and inert solvents.

In a tenth embodiment, the invention relates to a process according to any of the preceding embodiments, characterized in that the temperature
in the condensed-phase reaction is firstly between −40° C. and the boiling point of the solvent used or of the lowest-boiling input material at the pressure established in the reaction and
in the gas-phase phosgenation is between the boiling point of the reactant having the highest boiling point T1 at system pressure and 600° C., preferably between T1 and 550° C., more preferably between T1 and 500° C.

In an eleventh embodiment, the invention relates to a process according to any of the preceding embodiments, characterized in that the isocyanate corresponding to the first amine and/or the isocyanate corresponding to the second amine is separated from the reaction mixture, separation preferably being effected by distillation, in particular thin-film distillation, extraction, crystallization, recrystallization or a combination thereof, and with the respective isocyanates obtained separately or as an isocyanate mixture.

In a twelfth embodiment, the invention relates to a process according to any of embodiments 1 to 10, characterized in that the isocyanate corresponding to the first amine and/or the isocyanate corresponding to the second amine is separated from the reaction mixture, separation preferably being effected by distillation, in particular thin-film distillation, extraction, crystallization, recrystallization or a combination thereof, and with the respective isocyanates obtained as an isocyanate mixture.

In a thirteenth embodiment, the invention relates to a process according to embodiment 11 or 12, characterized in that the content of acidic chlorine compounds in the separated isocyanate(s) or in the isocyanate mixture is from 1 to 100 ppm, preferably from 2 to 80 ppm, more preferably from 5 to 50 ppm, determined according to the ISO 15028:2014 standard.

In a fourteenth embodiment, the invention relates to a process according to any of embodiments 11 to 13, characterized in that the content of hydrolyzable chlorine in the separated isocyanate(s) or in the isocyanate mixture is from 1 to 500 ppm, preferably from 5 to 100 ppm, determined according to the ISO 15028:2014 standard.

In a fifteenth embodiment, the invention relates to the use of an amine mixture formed of at least one first amine and at least one second amine different from the first amine in a process for producing the corresponding isocyanates by phosgenation according to a process according to any of embodiments 1 to 14 for reducing the content of acidic chlorine compounds and/or the content of hydrolyzable chlorine of the corresponding isocyanates obtained in the production process.

In a sixteenth embodiment, the invention relates to a product obtainable or obtained by a process according to any of embodiments 1 to 13, preferably according to any of embodiments 10 or 12 to 14, preferably directly obtainable or obtained by any of embodiments 1 to 13, preferably according to any of embodiments 10 or 12 to 14.

In a seventeenth embodiment, the invention relates to the use of the product according to embodiment 16 and/or of the isocyanate or isocyanate mixture obtained or obtainable by a process according to any of embodiments 11 to 14, preferably according to any of embodiments 10 or 12 to 14, as a component for the production of polyurethanes, in particular polyurethane foams, polyurethane coatings, and polyurethane adhesives, of pharmaceutical products, in particular active substances, and also of auxiliaries, in particular auxiliaries for wet-strength finishing of paper and other cellulose products, emulsifiers, and thickeners.

In an eighteenth embodiment, the invention relates to a phosgenation plant for performance of the process according to embodiments 1 to 14, comprising or consisting of:
(a) at least one phosgene apparatus for the continuous supply of phosgene, optionally in combination with an inert substance,
(b) optionally an inert substance apparatus for the supply of an inert substance,
(c) at least one amine apparatus for the continuous supply of an amine mixture, optionally in combination with an inert substance, and
(d) a phosgenation reactor for mixing the phosgene and the amine mixture and optionally the inert substance and reacting the amine mixture with the phosgene,
   characterized in that the amine apparatus comprises a first reservoir vessel for a first amine and a second reservoir vessel for a second amine, and also a metering device for independently variable adjustment of the mass fractions of the first amine and the second amine, and optionally a mixing device for mixing the first amine and the second amine to form the amine mixture.

In a nineteenth embodiment, the invention relates to an apparatus according to embodiment 18, characterized in that the amine apparatus, the optional separate inert substance apparatus and/or the phosgene apparatus include a heat exchanger for heating the amine mixture, the phosgene and/or optional inert substance.

### EXAMPLES

The examples described below are intended to describe the process and some of the process products of the invention more particularly without limiting the invention; all percentages are percentages by weight unless otherwise stated.

Mol % data were determined by ¹H-NMR spectroscopy. The measurements were carried out on Bruker DPX 400 or DRX 700 instruments on approx. 5% (¹H NMR) or approx. 50% (¹³C NMR) samples in dry C₆D₆, unless otherwise stated, at 400 or 700 MHz (¹H NMR) or 100 or 176 MHz (¹³C NMR). The reference employed for the ppm scale was tetramethylsilane in the solvent with ¹H-NMR chemical shift 0 ppm. Alternatively, C₆D₅H present in the NMR solvent was used as reference signal (7.15 ppm, ¹H-NMR), or the solvent signal itself (middle signal of the 1:1:1 triplet at 128.0 ppm in the ¹³C NMR. ¹⁵N-NMR chemical shifts were determined indirectly by ¹H-¹⁵N-HMBC measurements, which were referenced externally to (liquid) ammonia (0 ppm).

Dynamic viscosities were determined at 23° C. using the MCR 501 rheometer (from Anton Paar) in accordance with DIN EN ISO 3219:1994-10. Measurement at different shear rates ensured that Newtonian flow behavior can be assumed. Details regarding the shear rate can therefore be omitted.

The NCO content was determined by titration in accordance with DIN EN ISO 10283:2007-11.

The residual monomer content was determined by gas chromatography according to DIN EN ISO 10283:2007-11 with internal standard.

The content of acidic chlorine compounds and the content of hydrolyzable chlorine was determined in accordance with the ISO 15028:2014 standard.

GC-MS was carried out using the Agilent GC6890 equipped with an MN 725825.30 Optima-5 MS Accent capillary column (30 m, 0.25 mm internal diameter, 0.5 μm film layer thickness) and a 5973 mass spectrometer as detector with helium as transport gas (flow rate of 2 ml/min). The column temperature was initially 60° C. (2 min) and was then gradually increased to 360° C. at 8 K/min. The GC-MS detection employed electron-impact ionization with 70 eV ionization energy. An injector temperature of 250° C. was chosen.

Size-exclusion chromatography (SEC) was carried out in accordance with DIN 55672-1:2016-03 with tetrahydrofuran as eluent.

X-ray crystal structure analyses were carried out at 106-107 K on an Oxford Diffraction Xcalibur equipped with a CCD area detector (Ruby model), a Cu_{Kα} source, and Osmic mirrors as monochromator. The CrysAlis software program, version 1.171.38.43 (Rigaku 2015) was used for data acquisition and reduction. SHELXTL version 6.14 (Bruker AXS, 2003) was used for structural resolution.

The Hazen color number was measured spectrophotometrically in accordance with DIN EN ISO 6271-2:2005-03 using a Lico 400 spectrophotometer from Lange, Germany.

All reactions were carried out under a nitrogen atmosphere in glass apparatus dried beforehand under reduced pressure at 150-200° C.

The diisocyanates used are products from Covestro. All other commercially available chemicals were obtained from Aldrich, D-82018 Taufkirchen.

1,5-Diamino-3-oxapentane (OBDA) was obtained from BASF SE. TEFUDA, (3R,3aR,6S,6aR)-hexahydrofuro[3,2-b]furan-3,6-diamine (ISODA) and (3S,3aR,6S,6aR)-hexahydrofuro[3,2-b]furan-3,6-diamine (IDDA) were prepared from the corresponding hydroxyl compounds based on a literature method: S. Thiyagarajan, L. Gootjes, W. Vogelzang, J. van Haveren, M. Lutz, D. S. van Es ChemSusChem 2011, 4, 1823-1829).

### General Procedures

### A Liquid-Phase Phosgenations (LPP)

A 4-necked flask cooled to −5° C. with mechanical stirrer, dropping funnel, internal thermometer, and gas inlet tube was initially charged with 500 ml of monochlorobenzene (MCB) and then twice the molar amount of phosgene based on diamine (mixture) to be reacted was condensed in and the solution of the respective amine (mixture) was slowly added dropwise to the MCB, with further cooling at −10 to max. 0° C. After all the amine (mixture) had been added, the dropping funnel was filled with 100 ml of MCB and this was likewise added dropwise to the reaction mixture. While introducing phosgene (approx. 120 g/h) via the gas-inlet tube, the temperature was then gradually increased until MCB reflux occurred. On reaching the boiling temperature, stirring was continued under reflux with further passage of phosgene through the mixture (approx. 120 g/h) until a clear solution had formed, but for at least 5 hours. For dephosgenation, stirring was continued under reflux but passing dry nitrogen instead of phosgene through the mixture via the gas-inlet tube until absence of phosgene was ensured by phosgene indicator paper. The further workup is described in the respective examples.

### B Gas-Phase Phosgenations (GPP)

In a glass system having a heatable mixing tube 2.5 mm in diameter and 17.5 mm in length with a downstream condensation stage and subsequent phosgene adsorption tower filled with activated carbon, phosgene that had been preheated in an upstream heat exchanger was continuously introduced through a nozzle projecting into the mixing tube. This was accompanied by the simultaneous introduction into the reaction space, through the annular gap between the nozzle and the mixing tube, of a likewise preheated amine mixture at a metering rate optimized according to the particular amine (mixture), which was optionally diluted with dry nitrogen as diluent that was likewise preheated. The application of a reduced pressure at the end of the condensation stage allowed a defined pressure to be maintained in the mixing tube. The hot reaction mixture exiting the reaction space in gaseous form was conducted into a condensation stage by 1,2-dichlorobenzene boiling under reflux. This brought about the selective condensation of the diisocyanates that had formed. The gas mixture passing through the scrubbing stage, which consisted essentially of nitrogen, HCl, and excess phosgene, was subsequently freed of phosgene in the adsorption tower and the condensate freed of residual, dissolved phosgene as described under A. The further workup is described in the respective examples.

### Example 1 (Comparative Experiments)

a) Liquid-phase phosgenation of OBDA,
b) Co-phosgenation of OBDA with MDA in accordance with DE 2249459, and
b) Co-phosgenation of IDDA with MDA in accordance with DE 2249459.
   a)

20.83 g (0.2 mol) of OBDA was dissolved in approx. 400 ml of MCB and phosgenated according to general procedure A. The reaction mixture thus obtained was investigated by GC/GC-MS. In addition to the solvent, 3 main components in a ratio relative to one another of 3:1:47 were detected: 2-chloroethyl isocyanate, 2-(2-chloroethoxy)ethyl isocyanate, and OBDI. After removal of low boilers (monochlorobenzene and 2-chloroethyl isocyanate) under reduced pressure via a 40 cm column packed with #1 Interpack random packing, internal diameter approx. 25 mm, the remaining, largely solvent-free, distillation residue was then immediately subjected to a trap-to-trap distillation at 0.1 mbar and a bath temperature of 120-180° C. This afforded 10.5 g of a dark brown, highly viscous distillation residue having an NCO content of 10.8% and 15.2 g of a light-colored distillate having an NCO content of 53.0% and an AC/HC content of 220/560 ppm that according to NMR and GC-MS consisted to an extent of approx. 98% of OBDI and approx. 2% of 2-(2-chloroethoxy)ethyl isocyanate (approx. 50% yield of OBDI based on amine used). After a few days, the previously almost colorless liquid had discolored markedly. The product is unsuitable for further problem-free use in typical polyurethane applications.

b)

A mixture of 4.17 g (0.04 mol) of OBDA and 31.78 g of MDA was dissolved in approx. 400 ml of monochlorobenzene and phosgenated according to general procedure A. The reaction mixture thus obtained was investigated by GC/GC-MS. In addition to the solvent and aromatic isocyanates, 3 main components in a ratio relative to one another of 8.5:1:139 were detected: 2-chloroethyl isocyanate, 2-(2-chloroethoxy)ethyl isocyanate, and OBDI. After removal of low boilers (monochlorobenzene and 2-chloroethyl isocyanate) under reduced pressure via a 40 cm column packed with #1 Interpack random packing, internal diameter approx. 25 mm, the remaining, largely solvent-free, distillation residue was then immediately subjected to a trap-to-trap distillation at 0.1 mbar and a bath temperature of 120-180° C. This afforded 29.5 g of a dark brown, highly viscous distillation residue having an NCO content of 26.8% that was not analyzed further (mostly polymeric MDI) and 12.7 g of a yellowish distillate having an NCO content of 40.0% that according to NMR and GC-MS consisted to an extent of approx. 31.3% of OBDI, approx. 0.2% of 2-(2-chloroethoxy)ethyl isocyanate, and approx. 68.1% of 2-ring MDI (3 different isomers, mainly 4,4′-isomer) in addition to other, unidentified impurities (approx. 64% yield of OBDI based on OBDA used). The high content of cleavage products (2-chloroethyl isocyanate, 2-(2-chloroethoxy)ethyl isocyanate) and the low yield of OBDI demonstrate that the procedure according to DE 2249459 does not lead to success when using the first amine (amine 1).

c)

A mixture of 5.8 g (0.04 mol) of IDDA and 32.0 g of MDA was dissolved in approx. 1000 ml of monochlorobenzene and phosgenated according to general procedure A. The reaction mixture thus obtained was largely freed of solvent on a rotary evaporator under reduced pressure and then immediately subjected to a trap-to-trap distillation at 0.1 mbar and a bath temperature of 120-180° C. This afforded 20.8 g of a dark brown, highly viscous distillation residue having an NCO content of 28.0% that according to GPC and NMR mostly consisted of polymeric MDI and 27.6 g of a yellowish distillate that according to NMR and GC-MS consisted to an extent of approx. 25% of IDDI and approx. 75% of 2-ring MDI (3 different isomers, mainly 4,4′-isomer) in addition to other, unidentified impurities. Distillative separation of this mixture was unsuccessful. Even after repeated recrystallization from hot isooctane, residual amounts of aromatic diisocyanate remained in the IDDI. The procedure according to DE 2249459 does not lead to success when using the first amine (amine 1).

### Example 2 (Comparative Experiment) Preparation of IDDI from IDDA by LPP without the Second Amine

28.5 g (0.2 mol) of IDDA was phosgenated according to general procedure A. The reaction mixture thus obtained was largely freed of solvent on a rotary evaporator under reduced pressure and then immediately subjected to a trap-to-trap distillation at 0.1 mbar and a bath temperature of 120-180° C. This afforded 12.2 g of a dark brown, highly viscous distillation residue and 21.3 g of a light-colored distillate that crystallized after some time and that, according to a combination of analytical methods (NMR, GC-MS), consisted to an extent of approx. 98% of IDDI (53% yield based on amine used, AC/HC: 616/758 ppm). After storage at room temperature for a few days with exclusion of air, the previously almost colorless solid had discolored markedly. The product is unsuitable for further problem-free use in typical polyurethane applications.

### Example 3 (According to the Invention): CoPg-LPP of IDDA (First Amine) and PACM 20 (Second Amine)

33.7 g (0.16 mol) of PACM 20 and 5.8 g (0.04 mol) of IDDA were phosgenated according to general procedure A. The reaction mixture thus obtained was largely freed of solvent on a rotary evaporator under reduced pressure and then immediately subjected to a trap-to-trap distillation at 0.1 mbar and a bath temperature of 120-180° C. This afforded only 6.2 g of a dark brown, highly viscous distillation residue and 45.7 g of a light-colored distillate that after some time deposited proportions of crystalline material and that, according to a combination of analytical methods (NMR, GC-MS), consisted to an extent of approx. 13% of IDDI and 84% of H₁₂-MDI (78% and 91% yield based on amines used). Subsequent fractionation gave 5.6 g of a colorless, rapidly crystallizing distillate (boiling point 83° C. at 0.1 mbar, melting point 42° C.) that showed no tendency to discoloration even after weeks of storage at room temperature with exclusion of air and proved highly suitable for further problem-free use in typical polyurethane applications.

### Example 4 (Comparative Experiment)

Preparation of ISODI from ISODA by LPP without “second amine” 28.8 g (0.2 mol) of ISODA was phosgenated according to general procedure A. The reaction mixture thus obtained was largely freed of solvent on a rotary evaporator under reduced pressure and then immediately subjected to a trap-to-trap distillation at 0.1 mbar and a bath temperature of 120-180° C. This afforded 15.1 g of a dark brown, highly viscous distillation residue and 26.0 g (66% yield based on amine used) of a light-colored distillate that according to a combination of analytical methods (NMR, GC-MS) consisted to an extent of approx. 98% of ISODI. The titrated NCO content was 42.2% and the AC/HC values were 904/1548 ppm. After a few days, the previously almost colorless distillate had discolored markedly and after several weeks of storage at room temperature with exclusion of air was dark brown. The product is unsuitable for further problem-free use in typical polyurethane applications.

### Example 5 (According to the Invention): CoPg-LPP of IDDA (First Amine) and IPDA (Second Amine)

5.8 g (0.04 mol) of IDDA and 33.7 g (0.16 mol) of IPDA were phosgenated according to general procedure A. The reaction mixture thus obtained was largely freed of solvent on a rotary evaporator under reduced pressure and then immediately subjected to a trap-to-trap distillation at 0.1 mbar and a bath temperature of 120-180° C. This afforded only 3.6 g of a dark brown, highly viscous distillation residue and 40.6 g of a light-colored distillate that according to a combination of analytical methods (NMR, GC-MS) consisted to an extent of approx. 16.7% of ISODI and 83.3% of IPDI (86% and 95% yield based on amines used). The AC/HC content of this mixture was 42/132 ppm.

### Example 6 (Comparative Experiments)

Preparation of H₁₂-MDI from PACM 20 by GPP according to general procedure B (experiments a and b) and of IPDI from IPDA (experiment c)
a) In a gas-phase tubular reactor preheated to 350° C., 141 g of PACM 20 (0.67 mol) was reacted with 580 g of phosgene (5.86 mol) at 500 mbar over the course of 2 hours. The two material streams were both vaporized and heated to 350° C. at the same pressure in a heat exchanger. For better amine vaporization, 60 l/h of nitrogen was passed through the amine evaporator as a carrier gas stream. Immediately after the start of the reaction, the formation of crusts and deposits was clearly observed at the amine inlet and in the reaction tube. Accurate final weighing after the experiment revealed 8 g of solid.
b) In a gas-phase tubular reactor preheated to 450° C., 139 g of PACM 20 (0.66 mol) was reacted with 581 g of phosgene (5.87 mol) at 500 mbar over the course of 2 hours. The two material streams were both vaporized and heated to 450° C. at the same pressure in a heat exchanger. For better amine vaporization, 60 l/h of nitrogen was passed through the amine evaporator as a carrier gas stream. By comparison with example 6a, significantly less crusts and deposits formed. After the experiment there was only an extremely thin coating discernible on the wall of the reactor, which was determined by reweighing and amounted to 1.3 g.
c) In a gas-phase tubular reactor preheated to 350° C., 170 g of IPDA (1.0 mol) was reacted with 622 g of phosgene (6.29 mol) at 500 mbar over the course of 2 hours. The two material streams were both vaporized and heated to 350° C. at 500 mbar in a heat exchanger. For better amine vaporization, 30 l/h of nitrogen was passed through the amine evaporator. After the start of the reaction, only minimal formation of crusts and deposits was observed at the amine inlet and in the reaction tube; this was determined after the experiment and amounted to 0.5 g.

The reaction mixtures obtained under a and b were investigated by ¹H NMR, which found that, in addition to solvent and H₁₂-MDI, relevant proportions of an olefin were present, which were identified by GC-MS as cyclohexenylmethylcyclohexyl isocyanate (several isomers). In example 6b, the proportion of this undesirable by-product relative to the target compound H₁₂-MDI was significantly lower than in 6a: 67:1 compared to 4:1 (weight of H₁₂-MDI:weight of olefin).

Removal of solvent and subsequent trap-to-trap distillation as described in examples 2-5 afforded in a) 6.15 g of a dark brown, viscous distillation residue and 140.4 g of a light-colored distillate that, after subsequent fractionation via a 40 cm vacuum-jacketed Vigreux column having an internal diameter of approx. 25 mm, gave 123.6 g of a colorless main fraction having a constant boiling point and an NCO content of only 30.3% (<70% yield of H₁₂-MDI based on PACM 20 used). In b), 2.8 g of residue and 163 g of final distillate having an NCO content of 31.8% was obtained (93% yield of H₁₂-MDI based on PACM 20 used).

### Example 7 (According to the Invention): CoPg-GPP of TEFUDA (First Amine or Amine 1) and PACM 20 (Second Amine or Amine 2) by GPP According to General Procedure B

In accordance with example 6b, mixtures of TEFUDA (amine 1) and PACM 20 (amine 2) were phosgenated according to general procedure B. In this case, at the start of the experiment pure amine 2 (~0.335 mol/h) was metered in with a molar phosgene excess of about 250%. After an experiment time of 30 minutes, this was supplemented with amine 1 such that an amine mixture consisting of 25 mol % of amine 1 and 75 mol % of amine 2 was being metered in. In further experiments, the concentration of amine 1 was increased further such that amine mixtures containing 50 mol % amine 1/50 mol % amine 2 and 75 mol % amine 1/25 mol % amine 2 were used. In total, 782.6 g (6 mol) of TEFUDA and 1431.1 g (6.8 mol) of PACM 20 were processed. The amounts metered in, phosgene excesses used, and run times are given in the table below.

| | | | Amine | Phosgene | | Temperature | | | Run | Reactor |
|---|---|---|---|---|---|---|---|---|---|---|
| | Amine 1 | Amine 2 | amount | excess | Pressure | [° C.] | | | time | deposits |
| Example | [mol %] | [mol %] | [mol/h] | [mol %] | [mbara] | Amine | Phosgene | Reactor | [h] | [g] |
| 7-1 | 25 | 75 | 0.36 | 245 | 500 | 450 | 450 | 450 | 4.7 | 1.7 |
| 7-2 | 50 | 50 | 0.35 | 252 | 500 | 450 | 450 | 450 | 5.8 | 2.3 |
| 7-3 | 75 | 25 | 0.33 | 267 | 500 | 450 | 450 | 450 | 4.0 | 2.7 |
| 7-4 | 50 | 50 | 0.30 | 292 | 500 | 450 | 450 | 450 | 5.8 | 1.3 |
| 7-5 | 50 | 50 | 0.37 | 242 | 500 | 450 | 450 | 450 | 6.0 | 1.0 |
| 7-6 | 75 | 25 | 0.31 | 300 | 500 | 450 | 450 | 450 | 5.3 | 4.0 |
| 7-7 | 75 | 25 | 0.36 | 238 | 500 | 450 | 450 | 450 | 4.8 | 3.3 |

The dephosgenated crude products were investigated by ¹H-NMR spectroscopy and then combined. With regard to the H₁₂-MDI:olefin ratio, it was found that the proportion of the undesirable compound was by comparison with the better comparative example 6b again significantly reduced in all experimental runs 7-1 to 7-7 and averaged only 146:1. No significant differences in this ratio were observed in experimental runs 7-1 to 7-7.

Workup as described in example 6, with the difference that a more effective column was used for the final separation of TEFUDI and H₁₂-MDI (40 cm column packed with #1 Interpack random packing, internal diameter approx. 25 mm), gave only 53.9 g of a dark brown, highly viscous distillation residue and 980.5 g of TEFUDI and 1674.2 g of H₁₂-MDI, in each case as light-colored liquids that according to a combination of analytical methods (NMR, GC-MS) consisted to an extent of >99% of TEFUDI or H12-MDI (isomer mixture) (90% and 94% yield respectively based on amines used). The AC/HC content was 35/318 ppm (TEFUDI) and 29/87 ppm (H₁₂-MDI). Discoloration was not observed even after weeks of storage with exclusion of air.

### Example 8 (According to the Invention): CoPg-GPP of ISODA (First Amine or Amine 1) and PACM 20 (Second Amine or Amine 2) by GPP According to General Procedure B

In accordance with the procedure from example 7, mixtures of ISODA (amine 1) and PACM 20 (amine 2) were phosgenated in a gas-phase phosgenation apparatus. In experiment 8-1, at the start of the experiment pure amine 2 (~0.5 mol/h) was metered in with an approx. 250% molar excess of phosgene. After an experiment time of 30 minutes, this was supplemented with amine 1 such that an amine mixture consisting of 25 mol % of amine 1 and 75 mol % of amine 2 was being metered in. A total of two such stepwise increases by 25 mol % were carried out, in each case after a further 30 minutes, such that 2 hours after the start of the experiment an amine mixture composed of 75 mol % of amine 1 and 25 mol % of amine 2 was being metered in, which was then kept constant until the end of the experiment after a total run time of 6.25 hours. In the subsequent experiments, after the first phase with pure amine 2 an amine mixture consisting of 50 mol % of amine 1 and 50 mol % of amine 2 was immediately metered in, which was maintained until the end of the experiment. In addition, the phosgene excess was reduced from one experiment to the next, which was accompanied by a trend toward less reactor deposits. The amounts added in the individual experiments, phosgene excesses used, and run times are given in the table below. In total, 1353.9 g (9.39 mol) of ISODA and 915.0 g (4.35 mol) of PACM 20 were processed.

| | | | Amine | Phosgene | | Temperature | | | Run | Reactor |
|---|---|---|---|---|---|---|---|---|---|---|
| | Amine 1 | Amine 2 | amount | excess | Pressure | [° C.] | | | time | deposits |
| Example | [mol %] | [mol %] | [mol/h] | [mol %] | [m bara] | Amine | Phosgene | Reactor | [h] | [g] |
| 8-1 | 75 | 25 | 0.42 | 265 | 500 | 450 | 450 | 450 | 6.3 | 2.6 |
| 8-2 | 75 | 25 | 0.41 | 230 | 500 | 450 | 450 | 450 | 6.8 | 1.8 |
| 8-3 | 75 | 25 | 0.49 | 160 | 500 | 450 | 450 | 450 | 7.5 | 1.6 |
| 8-4 | 75 | 25 | 0.53 | 139 | 500 | 450 | 450 | 450 | 6.0 | 0.7 |
| 8-5 | 75 | 25 | 0.53 | 137 | 500 | 450 | 450 | 450 | 4.3 | 0.8 |

The dephosgenated crude products were investigated by ¹H-NMR spectroscopy and then combined. With regard to the H₁₂-MDI:olefin ratio, it was found that the proportion of the undesirable compound was by comparison with the better comparative example 6b again significantly reduced and averaged only 127:1. Here too, no significant differences in this ratio were observed in experimental runs 8-1 to 8-5.

Workup as described in example 7 gave only 60.1 g of a dark brown, highly viscous distillation residue and 1795.8 g of ISODI and 993.1 g of H₁₂-MDI as light-colored liquids that according to a combination of analytical methods (NMR, GC-MS) consisted to an extent of >99% of ISODI or H₁₂-MDI (isomer mixture) (97.5% and 87.0% yield based on amines used). The AC/HC content was 27/178 ppm (ISODI) and 15/152 ppm (H₁₂-MDI). Discoloration was not observed in either product obtained according to the invention even after weeks of storage at room temperature with exclusion of air.

### Example 9

a) Two comparative experiments of GPP of OBDA
b) According to the invention: CoPg-GPP of OBDA (first amine) and IPDA (second amine) by GPP according to general procedure B
   a)

In two separate experiments, OBDA and phosgene were reacted at 500 mbar in a gas-phase tubular reactor preheated to 350° C. The two material streams were both vaporized and heated to 350° C. at 500 mbar in a heat exchanger. For better amine vaporization, 30 l/h of nitrogen was passed through the amine evaporator as a carrier gas. In the first comparison experiment, 325 g of OBDA (3.1 mol) was reacted with 1376 g of phosgene (13.9 mol) over the course of 3.8 hours. In the second comparison experiment, 312 g of OBDA (3.0 mol) and 1219 g of phosgene (12.3 mol) were metered in over the course of 3.5 hours, in which reactor deposits amounting to 9 g were determined after the experiment.

Workup as described in example 6 gave 185 g of a dark brown, highly viscous distillation residue and 723.0 g of OBDI as a light-colored liquid that according to a combination of analytical methods (NMR, GC-MS) consisted to an extent of >98% of OBDI (76% yield based on amine used). The AC/HC content was 90/793 ppm. In the course of several weeks of storage at room temperature with exclusion of air, the product underwent significant discoloration. The color number increased from an initial 8 APHA immediately after distillative workup to 520 APHA after 3 weeks.

b)

In accordance with example 6c, an amine mixture consisting of 118.4 g of OBDA (1.14 mol) and 355.3 g of IPDA (2.09 mol) was metered over the course of 6 hours into a gas-phase tubular reactor preheated to 350° C. and reacted with 2223 g of phosgene (22.47 mol) at 500 mbar system pressure. The two material streams were both vaporized and heated to 350° C. at 500 mbar in a heat exchanger. For better amine vaporization, 30 l/h of nitrogen was passed through the amine evaporator. Downstream of the reactor, the reaction mixture was quenched in boiling o-dichlorobenzene and dephosgenated by nitrogen stripping. The formation of solid material at the amine inlet and in the reaction tube was determined after the experiment and amounted to 4 g.

Workup as described in example 6 gave only 12 g of a dark brown, highly viscous distillation residue, 169 g of OBDI, and 442 g of IPDI as light-colored liquids that according to a combination of analytical methods (NMR, GC-MS) consisted to an extent of >99% of OBDI or IPDI (isomer mixture) (in each case 95% yield based on amines used). The AC/HC content was 19/114 ppm (OBDI) and 12/212 ppm (IPDI). Discoloration was not observed in either product obtained according to the invention even after weeks of storage at room temperature with exclusion of air.

### Example 10 (According to the Invention): CoPg-GPP of ISODA (First Amine) and OBDA (Second Amine)

42.2 g of OBDA (0.4 mol) and 152 g of phosgene (1.54 mol) were first metered over the course of 30 minutes into a gas-phase tubular reactor preheated to 350° C. An amine mixture consisting of 54.0 g of ISODA (0.37 mol) and 117.3 g of OBDA (1.13 mol) was then metered in over the course of 3 hours and reacted with 933 g of phosgene (9.43 mol) at 500 mbar system pressure. The two material streams were both vaporized and heated to 350° C. at 500 mbar in a heat exchanger. For better amine vaporization, 60 l/h of nitrogen was passed through the amine evaporator as a carrier gas stream. Downstream of the reactor, the reaction mixture was quenched in boiling o-dichlorobenzene and dephosgenated by nitrogen stripping. The formation of solids at the amine inlet and in the reaction tube was determined after the experiment and amounted to 8.3 g.

Workup as described in example 6 gave only 16.6 g of a dark brown, highly viscous distillation residue, 49.8 g of ISODI, and 199.9 g of OBDI as light-colored liquids that according to a combination of analytical methods (NMR, GC-MS) consisted to an extent of >99% of ISODI or OBDI (68% and 84% yield respectively based on amines used). Discoloration was not observed in either product obtained according to the invention even after weeks of storage at room temperature with exclusion of air.

A number of attempts to react TEFUDA, IDDA or ISODA by GPP without any “amine 2” failed after a maximum run time of sixty minutes. Persistent caking formed and the pressure rise in the system made it necessary to shut down after an unacceptably short run time. The ISODI produced in very poor yield in these runs was of completely unacceptable quality and could be purified only extremely laboriously.

1. A process for producing isocyanates by phosgenation of amines, wherein an amine mixture formed of at least one first amine and at least one second amine different from the first amine is reacted with phosgene to provide a reaction mixture,
and wherein either
i) the first amine comprises at least one ether group; and
the second amine comprises aminoalkanes that have 5 to 15 hydrocarbon atoms and does not comprise ether groups;
or
ii) the first amine comprises at least one cyclic ether group; and
the second amine comprises at least one open-chain ether group and does not comprise cyclic ether groups.

2. The process in of claim 1, wherein the first amine comprises at least one ether group and includes a structure of the general formula X—(—R¹—NHₘ)ₙ, wherein
X is H, NHₘ or C(R²)p;
R¹ is optionally substituted or heteroatom-containing aliphatic, (cyclo)aliphatic or aromatic radicals having up to 10 carbon atoms, where R¹ contains at least one ether group;
R² is H or optionally substituted or heteroatom-containing aliphatic, (cyclo)aliphatic or aromatic radicals having up to 10 carbon atoms;
m is 1 or 2;
n is 1, 2, or 3; and
p is 1, 2 or 3.

3. The process of claim 1, wherein the first amine or the second amine is a diamine.

4. The process of claim 1, wherein the first amine is (tetrahydrofuran-2,5-diyl)dimethanamine, 3,6-diaminohexahydrofuro[3,2-b]furan, 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine, di(aminoethyl) ether, 2,2′-oxybis(ethan-1-amine), 1,1′-oxybis(propan-2-amine), 2-(2-aminoethoxy)propan-1-amine, 2,2′-oxybis(propan-1-amine), 2-(2-aminopropoxy)propan-1-amine, di(aminopropyl) ether or mixtures thereof.

5. The process of claim 1, wherein the second amine is 1,5-diaminopentane, 1,6-diaminohexane, 1-amino-3,5,5-trimethyl-5aminomethylcyclohexane, amino-[(aminocyclohexyl)methyl]cyclohexane, 4,4′-methylenebis(cyclohexan-1-amine), 2-((4-aminocyclohexyl)methyl)cyclohexan-1-amine, 2,2′-methylenebis(cyclohexane-1-amine), 1,3-bis(aminomethyl)cyclohexane, 1,4-bis(aminomethyl)cyclohexane, diaminocyclohexane, cyclohexane-1,2-diamine, cyclohexane-1,3-diamine, cyclohexane-1,4-diamine, methyldiaminocyclohexane, 4-methylcyclohexane-1,3-diamine, 2-methylcyclohexane-1,3-diamine, 4,4′-methylenebis(2-methylcyclohexan-1-amine) or mixtures thereof.

6. The process of claim 1, wherein the second amine is di(aminoethyl) ether, di(aminopropyl) ether, 1,8-diamino-1,5,8-trimethyl-3,6-dioxaoctane, 1,11-diamino-1,5,8,11-tetramethylundecane, 1,8-diamino-3,6-dioxaoctane, 1,10-diamino-4,7-dioxadecane, 1,12-diamino-4,9-dioxadodecane, 1,14-diamino-3,10-dioxatetradecane, 1,13-diamino-4,7,10-trioxatridecanane, 1,7-diamino-2,6-dioxa-4-aminomethoxy-heptane, 1-amino-2-oxa-3,3-bis(aminomethoxy)hexane, 1,9-diamino-3,7-dioxa-5-(1-amino-2-ethoxy)-nonane, 1-amino-3-oxa-4,4-bis(1-amino-2-ethoxy)heptane, 1,11-diamino-4,8-dioxa-6-(1-amino-5-oxabutyl)undecane, 1-amino-4-oxa-5,5-bis(1-amino-5-oxabutyl)octane or mixtures thereof.

7. The process of claim 1, wherein the mass fraction
of the first amine is 10.0% to 80.0% by weight and
of the second amine is 80.0% to 20.0%,
based on the total weight of the amine mixture.

8. The process of claim 1, wherein the reaction mixture further comprises an inert substance comprising:
inert gases;
inert solvents;
or mixtures thereof.

9. The process of claim 1, further comprising separating the isocyanates produced from the first amine or the isocyanates produced from the second amine, from the reaction mixture, and with the isocyanates are obtained separately or as an isocyanate mixture.

10. The process of claim 1, further comprising separating the isocyanates produced from the first amine or the isocyanates produced from the second amine, from the reaction mixture, wherein the separating is done by distillation, extraction, crystallization, recrystallization or a combination thereof, and the isocyanates are obtained as an isocyanate mixture.

11. The process of claim 9, wherein the content of acidic chlorine compounds in the separated isocyanates is from 1 to 100 ppm, determined according to the ISO 15028:2014 standard.

12-13. (canceled)

14. A polyurethane foam, coating, or adhesives comprising a polyurethane produced from the isocyanate produced from the process of claim 1.

15. A phosgenation plant for performing the process of claim 1, comprising:
(a) at least one phosgene apparatus for the continuous supply of phosgene, optionally in combination with an inert substance,
(b) optionally an inert substance apparatus for the supply of an inert substance,
(c) at least one amine apparatus for the continuous supply of an amine mixture, optionally in combination with an inert substance, and
(d) a phosgenation reactor for mixing the phosgene and the amine mixture and optionally the inert substance and reacting the amine mixture with the phosgene,
wherein the amine apparatus comprises a first reservoir vessel for a first amine and a second reservoir vessel for a second amine, and also a metering device for independently variable adjustment of the mass fractions of the first amine and the second amine, and optionally a mixing device for mixing the first amine and the second amine to form the amine mixture.

16. The process of claim 1, wherein the first amine is (tetrahydrofuran-2,5-diyl)dimethanamine, 3,6-diaminohexahydrofuro[3,2-b]furan, 3,6-bis(aminomethyl)hexahydrofuro[3,2-b]furan, furan-2,5-diyldimethanamine, (methane-2,2-diylbis(tetrahydrofuran-5,2-diyl))dimethanamine, (methane-2,2-diylbis(furan-5,2-diyl))dimethanamine, (propane-2,2-diylbis(tetrahydrofuran-5,2-diyl))dimethanamine, (propane-2,2-diylbis(furan-5,2-diyl))dimethanamine or mixtures thereof.

17. The process of claim 1, wherein the molar ratio of phosgene to the amino groups of the amines in the amine mixture is ≥1:1 to ≤5:1.

18. The process of claim 1, wherein the molar ratio of phosgene to the amino groups of the amines in the amine mixture is >1:1 to ≤3:1.

19. The process of claim 9, wherein the content of acidic chlorine compounds in the separated isocyanates is from 2 to 80 ppm, determined according to the ISO 15028:2014 standard.

20. The process of claim 9, wherein the content of acidic chlorine compounds in the separated isocyanates is from 5 to 50 ppm, determined according to the ISO 15028:2014 standard.

21. The process of claim 9, wherein the content of hydrolyzable chlorine in the separated isocyanates is from 1 to 500 ppm, determined according to the ISO 15028:2014 standard.

22. The process of claim 9, wherein the content of hydrolyzable chlorine in the separated isocyanates is from 5 to 100 ppm, determined according to the ISO 15028:2014 standard.
